(19) United States
(12) Patent Application Publication
(10) Pub. No.: US 2007/0093473 A1
(43) Pub. Date: Apr. 26, 2007

(54) 7-Heteroarylsulfonyl-tetrahydro-3-benzazepine derivatives as antipsychotic agents

(75) Inventors: David Gwyn Cooper, Essex (GB); Ian Thomson Forbes, Essex (GB); Vincenzo Garzya, Essex (GB); Andrew Derrick Gribble, Essex (GB); Andrew P. Lightfoot, Essex (GB); Andrew H. Payne, Essex (GB); Alexander Baxter Smith, Essex (GB)

Correspondence Address: SMITHKLINE BEECHAM CORPORATION;CORPORATE INTELLECTUAL PROPERTY-US, UW2220, P. O. BOX 1539, KING OF PRUSSIA, PA, 19406-0939, US

(73) Assignee: Glaxo Group Limited

(21) Appl. No.: 10/571,220

(22) PCT Filed: Sep. 9, 2004
(86) PCT No.: PCT/EP04/10131

§ 371 Date: Jan. 12, 2007

(30) Foreign Application Priority Data
Sep. 12, 2003 (GB) 0321475.6

(51) Int. Cl. A61K 31/55; C07D 403/02; C07D 409/02

(52) U.S. Cl. 514/217.01; 540/594

(57) ABSTRACT

The invention provides compounds of formula (I): 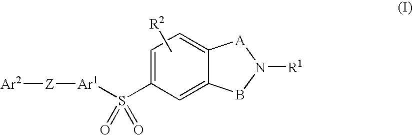 wherein
A and B represent the groups —(CH₂)ₘ— and —(CH₂)ₙ— respectively;
R¹ represents hydrogen or C₁₋₆alkyl;
R² represents hydrogen, halogen, hydroxy, cyano, nitro, hydroxyC₁₋₆alkyl, trifluoromethyl, trifluoromethoxy, C₁₋₆alkyl, C₁₋₆alkoxy, C₁₋₆fluoroalkoxy, —(CH₂)ₚC₃₋₆cycloalkyl, —(CH₂)ₚOC₃₋₆cycloalkyl, —COC₁₋₆alkyl, —SO₂C₁₋₆alkyl, —SOC₁₋₆alkyl, —S—C₁₋₆alkyl, —CO₂C₁₋₆alkyl, —CO₂NR³R⁴, —SO₂NR³R⁴, —(CH₂)ₚNR³R⁴, —(CH₂)ₚNR³COR⁴, optionally substituted aryl ring, optionally substituted heteroaryl ring, a fused bicyclic heteroaromatic ring system or optionally substituted heterocyclyl ring;
Ar¹ represents optionally substituted heteroaryl ring;
Ar² represents optionally substituted aryl ring or optionally substituted heteroaryl ring;
Z represents —(CH₂)_{q}X— wherein the —(CH₂)_{q}— group is attached to Ar², or —X(CH₂)_{q}— wherein X is attached to Ar², and wherein any of the —CH₂— groups may be optionally substituted by one or more C₁₋₆alkyl groups;
X represents oxygen, —CH(OR⁵)—, —NR⁵— or —CH₂— wherein the —CH₂— group may be optionally substituted by one or more C₁₋₆alkyl groups;
R³ and R⁴ each independently represent hydrogen, C₁₋₆alkyl or, together with the nitrogen or other atoms to which they are attached, form an azacycloalkyl ring or an oxo-substituted azacycloalkyl ring;
R⁵ represents hydrogen or C₁₋₆alkyl; m and n independently represent an integer selected from 1 and 2; p independently represents an integer selected from 0, 1, 2 and 3; q independently represents an integer selected from 0, 1, 2 and 3; or a pharmaceutically acceptable salt or solvate thereof,
with the proviso that when Ar¹ is a pyridyl group, and Z is —CH₂X— where X is attached to the Ar¹ group, X is selected from —CH(OR⁵)—, —NR⁵— and —CH₂— wherein the —CH₂— group may be optionally substituted by one or more C₁₋₆alkyl groups. The compounds of formula (I) are useful in therapy, in particular as antipsychotic agents.

This invention relates to novel compounds, pharmaceutical compositions containing them and their use in therapy, in particular as antipsychotic agents.

International patent application WO 02/85695 (BMS) discloses tetrahydroisoquinoline analogues useful as growth hormone secretagogues. Such analogues are also said to be useful in the treatment of disorders including inter alia, obesity, schizophrenia, depression and Alzheimer's disease.

International patent applications WO03/62205 and WO03/99786 (Glaxo Group Limited) disclose tetrahydroisoquinoline and tetrahydrobenzazepine derivatives which are said to be useful as antipsychotic agents.

We have now found a novel group of sulfonyl compounds which are useful particularly as antipsychotic agents.

According to the invention, there is provided a compound of formula (I): 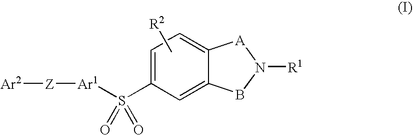
wherein

A and B represent the groups —(CH₂)ₘ— and —(CH₂)ₙ— respectively;

R¹ represents hydrogen or C₁₋₆alkyl;

R² represents hydrogen, halogen, hydroxy, cyano, nitro, hydroxyC₁₋₆alkyl, trifluoromethyl, trifluoromethoxy, C₁₋₆alkyl, C₁₋₆alkoxy, C₁₋₆fluoroalkoxy, —(CH₂)ₚC₃₋₆cycloalkyl, —(CH₂)ₚOC₃₋₆cycloalkyl, —COC₁₋₆alkyl, —SO₂C₁₋₆alkyl, —SOC₁₋₆alkyl, —S—C₁₋₆alkyl; —CO₂C₁₋₆alkyl, —CO₂NR³R⁴, —SO₂NR³R⁴, —(CH₂)ₚNR³R⁴, —(CH₂)ₚNR³COR⁴, optionally substituted aryl ring, optionally substituted heteroaryl ring, a fused bicyclic heteroaromatic ring system or optionally substituted heterocyclyl ring;

Ar¹ represents optionally substituted heteroaryl ring;

Ar² represents optionally substituted aryl ring or optionally substituted heteroaryl ring;

Z represents —(CH₂)_{q}X— wherein the —(CH₂)_{q}— group is attached to Ar² or —X(CH₂)_{q}— where in X is attached to Ar², and wherein any of the —CH₂— groups may be optionally substituted by one or more C₁₋₆alkyl groups;

X represents oxygen, —CH(OR⁵)—, —NR⁵— or —CH₂— wherein the —CH₂— group may be optionally substituted by one or more C₁₋₆alkyl groups;

R³ and R⁴ each independently represent hydrogen, C₁₋₆alkyl or, together with the nitrogen or other atoms to which they are attached, form an azacycloalkyl ring or an oxo-substituted azacycloalkyl ring;

R⁵ represents hydrogen or C₁₋₆alkyl;

m and n independently represent an integer selected from 1 and 2;

p independently represents an integer selected from 0, 1, 2 and 3;

q independently represents an integer selected from 0, 1, 2 and 3;

or a pharmaceutically acceptable salt or solvate thereof,

with the proviso that when Ar¹ is a pyridyl group, and Z is —CH₂X— where X is attached to the Ar¹ group, X is selected from —CH(OR⁵)—, —NR⁵— and —CH₂— wherein the —CH₂— group may be optionally substituted by one or more C₁₋₆alkyl groups.

When R² represents optionally substituted aryl ring, optionally substituted heteroaryl ring or optionally substituted heterocyclyl ring, the optional substituents may be independently selected from C₁₋₆alkyl, C₁₋₆alkoxy, halogen, trifluoromethyl, trifluoromethoxy, cyano, —S—C₁₋₆alkyl, —CONR⁵R⁶ and —NR⁵COR⁶, wherein R⁵ and R⁶ have any of the meanings given hereinbefore.

Ar¹ and Ar² may be optionally substituted by one or more substituents which may be the same or different, and which are selected from C₁₋₆alkyl, C₁₋₆alkoxy, halogen, trifluoromethyl, trifluoromethoxy, cyano, —S—C₁₋₆alkyl, —CONR⁵R⁶ and —NR⁵COR⁶, wherein R⁵ and R⁶ have any of the meanings given hereinbefore.

It is to be understood that the present invention covers all combinations of particular and preferred groups described herein above.

As used herein, the term “alkyl” refers to straight or branched hydrocarbon chains containing the specified number of carbon atoms. For example, C₁₋₆alkyl means a straight or branched alkyl containing at least 1, and at most 6, carbon atoms. Examples of “alkyl” as used herein include, but are not limited to, methyl, ethyl, n-propyl, n-butyl, n-pentyl, n-hexyl, isobutyl, isopropyl, t-butyl and 1,1-dimethylpropyl.

As used herein, the term “alkoxy” refers to a straight or branched alkoxy group containing the specified number of carbon atoms. For example, C₁₋₆alkoxy means a straight or branched alkoxy group containing at least 1, and at most 6, carbon atoms. Examples of “alkoxy” as used herein include, but are not limited to, methoxy, ethoxy, propoxy, prop-2-oxy, butoxy, but-2-oxy, 2-methylprop-1-oxy, 2-methylprop-2-oxy, pentoxy or hexyloxy.

As used herein, the term “fluoroalkoxy” refers to a straight or branched alkoxy group containing the specified number of carbon atoms wherein any of the carbon atoms may be substituted by one or more fluorine atoms.

As used herein, the term “cycloalkyi” refers to a non-aromatic hydrocarbon ring containing the specified number of carbon atoms. For example, C₃₋₇cycloalkyl means a non-aromatic ring containing at least three, and at most seven, ring carbon atoms. Examples of “cycloalkyl” as used herein include, but are not limited to, cyclopropyl, cyclobutyl, cyclopentyl, cyclohexyl and cycloheptyl. A C₆₋₇cycloalkyl group is preferred.

As used herein, the term “halogen” refers to the elements fluorine, chlorine, bromine and iodine. Preferred halogens are fluorine, chlorine and bromine.

As used herein, the term “aryl” refers to a phenyl or a naphthyl ring.

As used herein, the term “heteroaryl” refers to a monocyclic unsaturated ring containing at least one heteroatom independently selected from oxygen, nitrogen and sulfur. Examples of suitable 5- and 6-membered heterocyclic aromatic rings include, but are not limited to, furyl, thienyl, pyrrolyl, oxazolyl, thiazolyl, imidazolyl, oxadiazolyl, thiadiazolyl, pyridyl, triazolyl, tetrazolyl, triazinyl, pyridazinyl, pyrazinyl, pyrimidinyl, pyrazolyl, isothiazolyl and isoxazolyl.

As used herein, the term “heterocyclyl” refers to a 3- to 7-membered monocyclic saturated ring containing at least one heteroatom independently selected from oxygen, nitrogen and sulfur. Examples of suitable heterocyclic rings include, but are not limited to, piperidine and morpholine.

As used herein, the term “fused bicyclic heteroaromatic ring system” refers to a ring system comprising one six-membered unsaturated ring and one 5- or 6-membered unsaturated or saturated ring fused together, the ring system containing at least one heteroatom independently selected from oxygen, nitrogen and sulfur. Examples of suitable fused bicyclic heteroaromatic ring systems include, but are not limited to, indolyl, benzofuranyl, quinolyl and benzothienyl. Further examples include but are not limited to, isoquinolyl, quinolizinyl, naphthyridinyl, quinoxalinyl, quinazolinyl, cinnolinyl, isoindolyl, indolizinyl, indazolyl, pyrrolopyridinyl, benzimidazolyl, benzoxazolyl, benzisoxazolyl, benzothiazolyl, benzisothiazolyl, benzoxadiazolyl, benzothiadiazolyl, dihydrobenzothienyl, dihydrobenzofuranyl, benzodioxolanyl, methylenedioxyphenyl, dihydrobenzodioxinyl and the like.

As used herein, the term “azacycloalkyl ring” refers to a 4- to 7-membered monocyclic saturated ring containing one nitrogen atom. Examples of suitable azacycloalkyl rings are azetidine, pyrrolidine, piperidine and azepine.

As used herein, the term “oxo-substituted azacycloalkyl ring” refers to an azacyoloalkyl ring as defined above substituted by one oxo group. Examples of suitable oxo-substituted azacycloalkyl rings include, but are not limited to, azetidinone, pyrrolidinone, piperidinone and azepinone.

When Z represents —(CH₂)_{q}X— wherein the —(CH₂)_{q}— group is attached to Ar₂, examples of Z include —O—, —CH₂O—, —(CH₂)₂O—, —(CH₂)₃O—, —CH₂OR⁵—, —(CH₂)₂OR⁵—, —(CH₂)₃OR⁵—, —NR⁵—, —CH₂NR⁵, —(CH₂)₂NR⁵— and —(CH₂)₃NR⁵—.

When Z represents —X(CH₂)ᵣ wherein X is attached to Ar², examples of Z include —O—, —OCH₂, —O(CH₂)₂, —O(CH₂)₃, —OR⁵—CH₂—, —OR⁵—(CH₂)₂—, —OR⁵—(CH₂)₃, —NR⁵—, —NR⁵CH₂, —NR⁵(CH₂)₂— and —NR⁵(CH₂)₃—.

As used herein, the term “substituted” refers to substitution with the named substituent or substituents, multiple degrees of substitution being allowed unless otherwise stated.

As used herein, the term “solvate” refers to a complex of variable stoichiometry formed by a solute (in this invention, a compound of formula (I) or a salt thereof) and a solvent. Such solvents for the purpose of the invention may not interfere with the biological activity of the solute. Examples of suitable solvents include water, methanol, ethanol and acetic acid. Most preferably the solvent used is water and the solvate may also be referred to as a hydrate.

It will be appreciated that for use in medicine the salts of formula (I) should be physiologically (i.e. pharmaceutically) acceptable. Suitable physiologically acceptable salts will be apparent to those skilled in the art and include for example acid addition salts formed with inorganic acids e.g. hydrochloric, hydrobromic, sulfuric, nitric or phosphoric acid; and organic acids e.g. succinic, maleic, malic, mandelic, acetic, fumaric, glutamic, lactic, citric, tartaric, benzoic, benzenesulfonic, p-toluenesulfonic, methanesulfonic or naphthalenesulfonic acid. Other non-physiologically acceptable salts e.g. oxalates, may be used, for example in the isolation of compounds of formula (I) and are included within the scope of this invention. Also included within the scope of the invention are solvates and hydrates of the compounds of formula (I).

Certain of the compounds of formula (I) may form acid addition salts with one or more equivalents of the acid. The present invention includes within its scope all possible stoichiometric and non-stoichiometric forms thereof.

Certain compounds of formula (I) may exist in stereoisomeric forms (e.g. they may contain one or more asymmetric carbon atoms). The individual stereoisomers (enantiomers and diastereomers) and mixtures of these are included within the scope of the present invention. The present invention also covers the individual isomers of the compounds represented by formula (I) as mixtures with isomers thereof in which one or more chiral centres are inverted. Likewise, it is understood that compounds of formula (I) may exist in tautomeric forms other than that shown in the formula and these are also included within the scope of the present invention.

Preferably, R¹ represents hydrogen or C₁₋₄alkyl. More preferably, R¹ represents hydrogen, methyl, ethyl, n-propyl or isopropyl. Even more preferably, R¹ represents methyl.

The R² group may be located on any position on its phenyl ring.

Preferably, R² represents hydrogen, halogen, C₁₋₆alkyl, C₁₋₆alkoxy, C₁₋₆alkylthio or diC₁₋₆alkylamino. More preferably, R² represents hydrogen, halogen, C₁₋₄alkyl, C₁₋₄alkoxy or diC₁₋₆alkylamino. Even more preferably, R² represents methoxy.

In particular, Ar¹ represents optionally substituted thienyl or pyridyl (e.g. thien-2-yl or pyrid-3-yl). Preferably, Ar¹ represents optionally substituted thienyl. More preferably, Ar¹ is unsubstituted thienyl (e.g. 2-thienyl).

Preferably, Ar² represents optionally substituted phenyl. Preferably, the optional substituents on Ar² are independently selected from halogen. In particular, the optional substituents are independently selected from fluoro (e.g. 4-fluorophenyl or 3,4-difluorophenyl) or chloro (e.g. 4-chlorophenyl). More preferably, the optional substituents are selected from fluoro (e.g. 4-fluorophenyl).

Preferably, Z is —O—CH₂—, —O— or —CH—.

Preferably, R³ and R⁴ independently represent hydrogen or C₁₋₄alkyl.

Preferably, p represents 0 or 1.

Preferably, q represents 0 or 1.

In a first embodiment of the invention, the R² group is located at the para-position relative to the group B i.e. a compound of formula (IA) 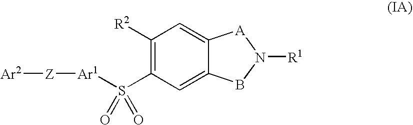
or a pharmaceutically acceptable salt or solvate thereof wherein groups A, B, Ar¹, Ar², Z, R¹ and R² have any of the meanings as given hereinbefore.

When R² is located in the para-position i.e. compounds of formula (IA), R² is preferably methoxy.

In another embodiment of the invention, m is 1 and n is 1 and the invention is a compound of formula (IB): 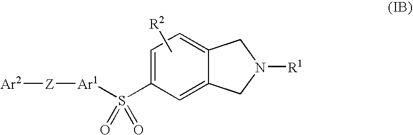
or a pharmaceutically acceptable salt or solvate thereof wherein groups Ar¹, Ar², Z, R¹ and R² have any of the meanings as given hereinbefore.

In another embodiment of the invention, m is 2 and n is 1 and the invention is a compound of formula (IC): 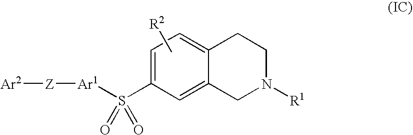
or a pharmaceutically acceptable salt or solvate therefor wherein groups Ar¹, Ar², Z, R¹ and R² have any of the meanings as given hereinbefore.

In another embodiment of the invention, m is 1 and n is 2 and the invention is a compound of formula (ID): 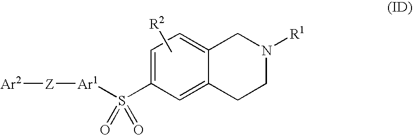
or a pharmaceutically acceptable salt or solvate thereof wherein groups Ar¹, Ar², Z, R¹ and R² have any of the meanings as given hereinbefore.

In another embodiment of the invention, m is 2 and n is 2 and the invention is a compound of formula (IE): 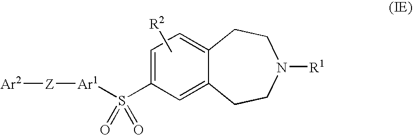
or a pharmaceutically acceptable salt or solvate thereof wherein groups Ar¹, Ar², Z, R¹ and R² have any of the meanings as given hereinbefore.

In another embodiment of the invention, m is 2 and n is 2, the R² group is located at the para-position relative to the group B and the invention is a compound of formula (IF): 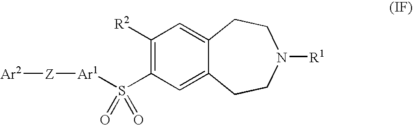
or a pharmaceutically acceptable salt or solvate thereof wherein groups Ar¹, Ar², Z, R¹ and R² have any of the meanings as given hereinbefore.

Particular compounds according to the invention include those incorporated in Table 1 and specifically exemplified and named hereinafter including, without limitation:

7-[(5-{[(4-Fluorophenyl)oxy]methyl}-2-thienyl)sulfonyl]-3-methyl-8-(methyloxy)-2,3,4,5-tetrahydro-1H-3-benzazepine;

7-({5-[(4-Chlorophenyl)oxy]-2-thienyl}sulfonyl)-3-methyl-8-(methyloxy)-2,3,4,5-tetrahydro-1H-3-benzazepine;

7-({5-[(4-Fluorophenyl)methyl]-2-thienyl}sulfonyl)-3-methyl-8-(methyloxy)-2,3,4,5-tetrahydro-1H-3-benzazepine;

7-[6-(4-Chlorophenoxymethyl)pyridine-3-sulfonyl]-8-methoxy-3-methyl-2,3,4,5-tetrahydro-1H-3-benzazepine;

7-[6-(3,4-Difluorophenoxymethyl)pyridine-3-sulfonyl]-8-methoxy-3-methyl-2,3,4,5-tetrahydro-1H-3-benzazepine,

and pharmaceutically acceptable salts and solvates thereof.

The compounds of the present invention may be in the form of their free base or physiologically acceptable salts thereof, particularly the monohydrochloride or monomesylate salts or pharmaceutically acceptable derivatives thereof.

The present invention also provides a general process (A) for preparing compounds of formula (I) which process comprises:
reacting a compound of formula (II) 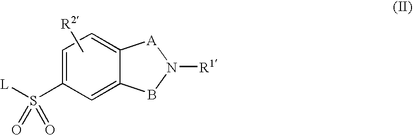
with a compound of formula (III)
Ar^{2′}-Z-Ar^{1′}-M (III)
wherein L is a leaving group, such as fluoro, chloro, alkoxy or aryloxy, M is a metal, such as lithium or magnesium, and R^{1′}, R^{2′}, Ar^{1′} and Ar^{2′} represent R¹, R², Ar¹ and Ar² as hereinbefore defined or are groups that may be readily convertible to R¹, R², Ar¹ and Ar², and A, B and Z are as hereinbefore defined.

This general method (A) can be conveniently performed by mixing the two components at preferably −70° C. to room temperature in a suitable solvent such as tetrahydrofuran or ether for 10 minutes to 18 hours. Removal of certain R^{1′} protecting groups e.g. trifluoroacetyl, can also take place simultaneously during this process.

Thus, in a further aspect of the invention, there is provided a process for the preparation of the compound of formula (I) as hereinbefore defined by:
a) reacting a compound of formula (II) 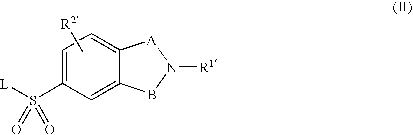
with a compound of formula (Ill)
Ar^{2′}-Z-Ar^{1′}-M (III)
wherein L is a leaving group, M is a metal, and R^{1′}, R², Ar^{1′} and Ar^{2″} represent R¹, R², Ar¹ and Ar² as hereinbefore defined or are groups that may be readily convertible to R¹, R², Ar¹ and Ar², and A, B and Z are as hereinbefore defined; and thereafter optionally for process (a):
removing any protecting groups; or
converting a compound of formula (I) into another compound of formula (I); or
forming a pharmaceutically acceptable salt.

Interconversion of one of the R^{1′} or R^{2′} groups to the corresponding R¹ to R² groups typically arises when one compound of formula (I) is used as the immediate precursor of another compound of formula (I), or when it is easier to introduce a more complex or reactive substituent at the end of a synthetic sequence.

For example, conversion of R^{1′} from a t-butoxycarbonyl (BOC) group to hydrogen is conducted by the treatment of the N—BOC protected compound with hydrogen chloride in ethanol or dioxan at room temperature.

Conversion of R^{1′} from hydrogen to an alkyl group is conducted by the treatment of the NH compound with the appropriate aldehyde in dichloroethane in the presence of a reducing agent, such as sodium triacetoxyborohydride, or by the treatment of the NH compound with the appropriate alkyl halide, such as iodomethane, under standard alkylation conditions (potassium carbonate in DMF at 60° C.).

Compounds of formula (II) are known in the literature or may be prepared by known processes, for example, chlorosulfonation of the aromatic ring using chlorosulfonic acid. Conversion to the sulfonyl fluoride can be achieved, if required, by reaction with potassium fluoride in acetonitrile at room temperature. Suitable examples of an R¹′ protecting group are trifluoroacetyl or the t-butoxycarbonyl (BOC) group.

Compounds of formula (III) are commercially available or may be prepared by established procedures, for example lithiation of the corresponding thiophene in tetrahydrofuran at low temperature, with for example t-butyl lithium.

Compounds of formula (I) have antagonist affinity for the serotonin 5-HT_{2C}, 5-HT_{2A} and 5-HT₆ receptors. These properties may give rise to anti-psychotic activity (e.g. improved effects on cognitive dysfunction), activity with reduced extrapyramidal side effects (eps), and/or anxiolytic/antidepressant activity. These could include, but are not limited to, attenuation of cognitive symptoms via 5-HT₆ receptor blockade (see Reavill, C. and Rogers, D. C., 2001, Investigational Drugs 2, 104-109), and reduced anxiety (see for example Kennett et al., Neuropharmacology 1997 Apr-May; 36 (4-5): 609-20), protection against EPS (Reavill et al., Brit. J. Pharmacol., 1999; 126: 572-574) and antidepressant activity (Bristow et al., Neuropharmacology 39:2000; 1222-1236) via 5-HT_{2C} receptor blockade.

Certain compounds of formula (I) have also been found to exhibit affinity for dopamine receptors, in particular the D₃ and D₂ receptors, and are useful in the treatment of disease states which require modulation of such receptors, such as psychotic conditions. Many of the compounds of formula (I) have also been found to have greater affinity for dopamine D₃ than for D₂ receptors. The therapeutic effect of currently available antipsychotic agents (neuroleptics) is generally believed to be exerted via blockade of D₂ receptors; however this mechanism is also thought to be responsible for undesirable eps associated with many antipsychotic agents. Without wishing to be bound by theory, it has been suggested that blockade of the dopamine D₃ receptor may give rise to beneficial antipsychotic activity without significant eps (see for example Sokoloff et al, Nature, 1990; 347: 146-151; and Schwartz et al, Clinical Neuropharmacology, Vol 16, No. 4, 295-314, 1993).

Compounds of formula (I) may also exhibit affinity for other receptors not mentioned above, resulting in beneficial antipyschotic activity.

Within the context of the present invention, the terms used herein are classified in the Diagnostic and Statistical Manual of Mental Disorders, 4^{th} Edition, published by the American Psychiatric Association (DSM-IV) and/or the International Classification of Diseases, 10^{th} Edition (ICD-10). The various subtypes of the disorders mentioned herein are contemplated as part of the present invention. Numbers in brackets after the listed diseases below refer to the classification code in DSM-IV.

The compounds of formula (I) are of use as antipsychotic agents for example in the treatment of schizophrenia, schizo-affective disorders, schizophreniform disorders, psychotic depression, mania, acute mania, paranoid and delusional disorders.

In particular, the compounds of formula (I) are of use in the treatment of schizophrenia including the subtypes Paranoid Type (295.30), Disorganised Type (295.10), Catatonic Type (295.20), Undifferentiated Type (295.90) and Residual Type (295.60); Schizophreniform Disorder (295.40); Schizoaffective Disorder (295.70) including the subtypes Bipolar Type and Depressive Type; Delusional Disorder (297.1) including the subtypes Erotomanic Type, Grandiose Type, Jealous Type, Persecutory Type, Somatic Type, Mixed Type and Unspecified Type; Brief Psychotic Disorder (298.8); Shared Psychotic Disorder (297.3); Psychotic Disorder Due to a General Medical Condition including the subtypes With Delusions and With Hallucinations; Substance-induced Psychotic Disorder including the subtypes With Delusions (293.81) and With Hallucinations (293.82); and Psychotic Disorder Not Otherwise Specified (298.9).

The compounds of formula (I) are also of use in the treatment of depression and mood disorders including Major Depressive Episode, Manic Episode, Mixed Episode and Hypomanic Episode; Depressive Disorders including Major Depressive Disorder, Dysthymic Disorder (300.4), Depressive Disorder Not Otherwise Specified (311); Bipolar Disorders including Bipolar I Disorder, Bipolar II Disorder (Recurrent Major Depressive Episodes with Hypomanic Episodes) (296.89), Cyclothymic Disorder (301.13) and Bipolar Disorder Not Otherwise Specified (296.80); Other Mood Disorders including Mood Disorder Due to a General Medical Condition (293.83) which includes the subtypes With Depressive Features, With Major Depressive—like Episode, With Manic Features and With Mixed Features), Substance-Induced Mood Disorder (including the subtypes With Depressive Features, With Manic Features and With Mixed Features) and Mood Disorder Not Otherwise Specified (296.90).

The compounds of formula (I) are also of use in the treatment of anxiety disorders including Social Anxiety Disorder, Panic Attack, Agoraphobia, Panic Disorder, Agoraphobia Without History of Panic Disorder (300.22), Specific Phobia (300.29) including the subtypes Animal Type, Natural Environment Type, Blood-Injection-Injury Type, Situational Type and Other Type), Social Phobia (300.23), Obsessive-Compulsive Disorder (300.3), Posttraumatic Stress Disorder (309.81), Acute Stress Disorder (308.3), Generalized Anxiety Disorder (300.02), Anxiety Disorder Due to a General Medical Condition (293.84), Substance-Induced Anxiety Disorder and Anxiety Disorder Not Otherwise Specified (300.00).

The compounds of formula (I) are also of use in the treatment of substance-related disorders including Substance Use Disorders such as Substance Dependence, Substance Craving and Substance Abuse; Substance-Induced Disorders such as Substance Intoxication, Substance Withdrawal, Substance-Induced Delirium, Substance-Induced Persisting Dementia, Substance-Induced Persisting Amnestic Disorder, Substance-Induced Psychotic Disorder, Substance-Induced Mood Disorder, Substance-Induced Anxiety Disorder, Substance-Induced Sexual Dysfunction, Substance-Induced Sleep Disorder and Hallucinogen Persisting Perception Disorder (Flashbacks); Alcohol-Related Disorders such as Alcohol Dependence (303.90), Alcohol Abuse (305.00), Alcohol Intoxication (303.00), Alcohol Withdrawal (291.81), Alcohol Intoxication Delirium, Alcohol Withdrawal Delirium, Alcohol-Induced Persisting Dementia, Alcohol-Induced Persisting Amnestic Disorder, Alcohol-Induced Psychotic Disorder, Alcohol-Induced Mood Disorder, Alcohol-Induced Anxiety Disorder, Alcohol-Induced Sexual Dysfunction, Alcohol-Induced Sleep Disorder and Alcohol-Related Disorder Not Otherwise Specified (291.9); Amphetamine (or Amphetamine-Like)-Related Disorders such as Amphetamine Dependence (304.40), Amphetamine Abuse (305.70), Amphetamine Intoxication (292.89), Amphetamine Withdrawal (292.0), Amphetamine Intoxication Delirium, Amphetamine Induced Psychotic Disorder, Amphetamine-Induced Mood Disorder, Amphetamine-Induced Anxiety Disorder, Amphetamine-Induced Sexual Dysfunction, Amphetamine-Induced Sleep Disorder and Amphetamine-Related Disorder Not Otherwise Specified (292.9); Caffeine Related Disorders such as Caffeine Intoxication (305.90), Caffeine-Induced Anxiety Disorder, Caffeine-lnduced Sleep Disorder and Caffeine-Related Disorder Not Otherwise Specified (292.9); Cannabis-Related Disorders such as Cannabis Dependence (304.30), Cannabis Abuse (305.20), Cannabis Intoxication (292.89), Cannabis Intoxication Delirium, Cannabis-Induced Psychotic Disorder, Cannabis-Induced Anxiety Disorder and Cannabis-Related Disorder Not Otherwise Specified (292.9); Cocaine-Related Disorders such as Cocaine Dependence (304.20), Cocaine Abuse (305.60), Cocaine Intoxication (292.89), Cocaine Withdrawal (292.0), Cocaine Intoxication Delirium, Cocaine-Induced Psychotic Disorder, Cocaine-Induced Mood Disorder, Cocaine-Induced Anxiety Disorder, Cocaine-Induced Sexual Dysfunction, Cocaine-Induced Sleep Disorder and Cocaine-Related Disorder Not Otherwise Specified (292.9); Hallucinogen-Related Disorders such as Hallucinogen Dependence (304.50), Hallucinogen Abuse (305.30), Hallucinogen Intoxication (292.89), Hallucinogen Persisting Perception Disorder (Flashbacks) (292.89), Hallucinogen Intoxication Delirium, Hallucinogen-Induced Psychotic Disorder, Hallucinogen-Induced Mood Disorder, Hallucinogen-Induced Anxiety Disorder and Hallucinogen-Related Disorder Not Otherwise Specified (292.9); Inhalant-Related Disorders such as Inhalant Dependence (304.60), Inhalant Abuse (305.90), Inhalant Intoxication (292.89), Inhalant Intoxication Delirium, Inhalant-Induced Persisting Dementia, Inhalant-Induced Psychotic Disorder, Inhalant-Induced Mood Disorder, Inhalant-Induced Anxiety Disorder and Inhalant-Related Disorder Not Otherwise Specified (292.9); Nicotine-Related Disorders such as Nicotine Dependence (305.1), Nicotine Withdrawal (292.0) and Nicotine-Related Disorder Not Otherwise Specified (292.9); Opioid-Related Disorders such as Opioid Dependence (304.00), Opioid Abuse (305.50), Opioid Intoxication (292.89), Opioid Withdrawal (292.0), Opioid Intoxication Delirium, Opioid-Induced Psychotic Disorder, Opioid-Induced Mood Disorder, Opioid-Induced Sexual Dysfunction, Opioid-Induced Sleep Disorder and Opioid-Related Disorder Not Otherwise Specified (292.9); Phencyclidine (or Phencyclidine-Like)-Related Disorders such as Phencyclidine Dependence (304.60), Phencyclidine Abuse (305.90), Phencyclidine Intoxication (292.89), Phencyclidine Intoxication Delirium, Phencyclidine-Induced Psychotic Disorder, Phencyclidine-Induced Mood Disorder, Phencyclidine-Induced Anxiety Disorder and Phencyclidine-Related Disorder Not Otherwise Specified (292.9); Sedative-, Hypnotic-, or Anxiolytic-Related Disorders such as Sedative, Hypnotic, or Anxiolytic Dependence (304.10), Sedative, Hypnotic, or Anxiolytic Abuse (305.40), Sedative, Hypnotic, or Anxiolytic Intoxication (292.89), Sedative, Hypnotic, or Anxiolytic Withdrawal (292.0), Sedative, Hypnotic, or Anxiolytic Intoxication Delirium, Sedative, Hypnotic, or Anxiolytic Withdrawal Delirium, Sedative-, Hypnotic-, or Anxiolytic-Persisting Dementia, Sedative-, Hypnotic-, or Anxiolytic-Persisting Amnestic Disorder, Sedative-, Hypnotic-, or Anxiolytic-Induced Psychotic Disorder, Sedative-, Hypnotic-, or Anxiolytic-Induced Mood Disorder, Sedative-, Hypnotic-, or Anxiolytic-Induced Anxiety Disorder Sedative-, Hypnotic-, or Anxiolytic-Induced Sexual Dysfunction, Sedative-, Hypnotic-, or Anxiolytic-Induced Sleep Disorder and Sedative-, Hypnotic-, or Anxiolytic-Related Disorder Not Otherwise Specified (292.9); Polysubstance-Related Disorder such as Polysubstance Dependence (304.80); and Other (or Unknown) Substance-Related Disorders such as Anabolic Steroids, Nitrate Inhalants and Nitrous Oxide.

The compounds of formula (I) are also of use in the treatment of sleep disorders including primary sleep disorders such as Dyssomnias such as Primary Insomnia (307.42), Primary Hypersomnia (307.44), Narcolepsy (347), Breathing-Related Sleep Disorders (780.59), Circadian Rhythm Sleep Disorder (307.45) and Dyssomnia Not Otherwise Specified (307.47); primary sleep disorders such as Parasomnias such as Nightmare Disorder (307.47), Sleep Terror Disorder (307.46), Sleepwalking Disorder (307.46) and Parasomnia Not Otherwise Specified (307.47); Sleep Disorders Related to Another Mental Disorder such as Insomnia Related to Another Mental Disorder (307.42) and Hypersomnia Related to Another Mental Disorder (307.44); Sleep Disorder Due to a General Medical Condition; and Substance-Induced Sleep Disorder including the subtypes Insomnia Type, Hypersomnia Type, Parasomnia Type and Mixed Type.

Furthermore, they may have utility as adjunct therapy in Parkinson's Disease, particularly with compounds such as L-DOPA and possibly dopaminergic agonists, to reduce the side effects experienced with these treatments on long term use (e.g. see Schwartz et al., Brain Res. Reviews, 1998, 26, 236-242). From the localisation of D₃ receptors, it could also be envisaged that the compounds could also have utility for the treatment of substance abuse where it has been suggested that D3 receptors are involved (e.g. see Levant, 1997, Pharmacol. Rev., 49, 231-252). Examples of such substance abuse include alcohol, cocaine, heroin and nicotine abuse. Other conditions which may be treated by the compounds include dyskinetic disorders such as Parkinson's disease, neuroleptic-induced parkinsonism and tardive dyskinesias; depression; anxiety; agitation; tension; social or emotional withdrawal in psychotic patients; cognitive impairment including memory disorders such as Alzheimer's disease; psychotic states associated with neurodegenerative disorders, e.g. Alzheimer's disease; eating disorders; obesity; sexual dysfunction; sleep disorders; emesis; movement disorders; obsessive-compulsive disorders; amnesia; aggression; autism; vertigo; dementia; circadian rhythm disorders; convulsions; epilepsy; and gastric motility disorders e.g. IBS.

The compounds of formula (I) are also of use in the treatment of eating disorders such as Anorexia Nervosa (307.1) including the subtypes Restricting Type and Binge-Eating/Purging Type; Bulimia Nervosa (307.51) including the subtypes Purging Type and Nonpurging Type; Obesity; Compulsive Eating Disorder; and Eating Disorder Not Otherwise Specified (307.50).

The compounds of formula (I) are also of use in the treatment of Autistic Disorder (299.00); Attention-Deficit/Hyperactivity Disorder including the subtypes Attention-Deficit/Hyperactivity Disorder Combined Type (314.01), Attention-Deficit/Hyperactivity Disorder Predominantly Inattentive Type (314.00), Attention-Deficit/Hyperactivity Disorder Hyperactive-Impulse Type (314.01) and Attention-Deficit/Hyperactivity Disorder Not Otherwise Specified (314.9); Hyperkinetic Disorder; Disruptive Behaviour Disorders such as Conduct Disorder including the subtypes childhood-onset type (321.81), Adolescent-Onset Type (312.82) and Unspecified Onset (312.89), Oppositional Defiant Disorder (313.81) and Disruptive Behaviour Disorder Not Otherwise Specified; and Tic Disorders such as Tourette's Disorder (307.23).

The compounds of formula (I) are also of use in the treatment of Personality Disorders including the subtypes Paranoid Personality Disorder (301.0), Schizoid Personality Disorder (301.20), Schizotypal Personality Disorder (301.22), Antisocial Personality Disorder (301.7), Borderline Personality Disorder (301.83), Histrionic Personality Disorder (301.50), Narcissistic Personality Disorder (301.81), Avoidant Personality Disorder (301.82), Dependent Personality Disorder (301.6), Obsessive-Compulsive Personality Disorder (301.4) and Personality Disorder Not Otherwise Specified (301.9).

The compounds of formula (I) are also of use for enhancement of cognition or for the treatment of cognition impairment, including the treatment of cognition impairment in other diseases such as schizophrenia, bipolar disorder, depression and other psychiatric disorders.

The compounds of formula (I) are also of use in the treatment of sexual dysfunctions including Sexual Desire Disorders such as Hypoactive Sexual Desire Disorder (302.71), and Sexual Aversion Disorder (302.79); sexual arousal disorders such as Female Sexual Arousal Disorder (302.72) and Male Erectile Disorder (302.72); orgasmic disorders such as Female Orgasmic Disorder (302.73), Male Orgasmic Disorder (302.74) and Premature Ejaculation (302.75); sexual pain disorder such as Dyspareunia (302.76) and Vaginismus (306.51); Sexual Dysfunction Not Otherwise Specified (302.70); paraphilias such as Exhibitionism (302.4), Fetishism (302.81), Frotteurism (302.89), Pedophilia (302.2), Sexual Masochism (302.83), Sexual Sadism (302.84), Transvestic Fetishism (302.3), Voyeurism (302.82) and Paraphilia Not Otherwise Specified (302.9); gender identity disorders such as Gender Identity Disorder in Children (302.6) and Gender Identity Disorder in Adolescents or Adults (302.85); and Sexual Disorder Not Otherwise Specified (302.9).

Therefore, the invention provides a compound of formula (I) as hereinbefore described or a pharmaceutically acceptable salt or solvate thereof for use in therapy.

The invention also provides a compound of formula (I) or a pharmaceutically acceptable salt or solvate thereof for use in the treatment of a condition which requires modulation of a dopamine receptor.

The invention also provides a compound of formula (I) as hereinbefore described or a pharmaceutically acceptable salt or solvate thereof for use in the treatment of schizophrenia, mood disorders, anxiety disorders, substance-related disorders, sleep disorders, eating disorders, autistic disorder, attention-deficit/hyperactivity disorder, disruptive behaviour disorder, tic disorders, personality disorders, cognition impairment in other diseases, sexual dysfunction, Parkinson's disease, dyskinetic disorders, depression, bipolar disorder, cognitive impairment, obesity, emesis, movement disorders, obsessive-compulsive disorders, amnesia, aggression, vertigo, dementia and circadian rhythm disorders.

The invention also provides a compound of formula (I) as hereinbefore described or a pharmaceutically acceptable salt or solvate thereof for use in the treatment of psychotic disorders, schizophrenia, Parkinson's disease, substance abuse, dyskinetic disorders, depression, bipolar disorder, anxiety, cognitive impairment, eating disorders, obesity, sexual dysfunction, sleep disorders, emesis, movement disorders, obsessive-compulsive disorders, amnesia, aggression, autism, vertigo, dementia, circadian rhythm disorders and gastric motility disorders.

The invention also provides the use of a compound of formula (I) as hereinbefore described or a pharmaceutically acceptable salt or solvate thereof in the manufacture of a medicament for the treatment of a condition which requires modulation of a dopamine receptor.

The invention also provides the use of a compound of formula (I) as hereinbefore described or a pharmaceutically acceptable salt or solvate thereof in the manufacture of a medicament for the treatment of schizophrenia, mood disorders, anxiety disorders, substance-related disorders, sleep disorders, eating disorders, autistic disorder, attention-deficit/hyperactivity disorder, disruptive behaviour disorder, tic disorders, personality disorders, cognition impairment in other diseases, sexual dysfunction, Parkinson's disease, dyskinetic disorders, depression, bipolar disorder, cognitive impairment, obesity, emesis, movement disorders, obsessive-compulsive disorders, amnesia, aggression, vertigo, dementia and circadian rhythm disorders.

The invention also provides the use of a compound of formula (I) as hereinbefore described or a pharmaceutically acceptable salt or solvate thereof in the manufacture of a medicament for the treatment of psychotic disorders, schizophrenia, Parkinson's disease, substance abuse, dyskinetic disorders, depression, bipolar disorder, anxiety, cognitive impairment, eating disorders, obesity, sexual dysfunction, sleep disorders, emesis, movement disorders, obsessive-compulsive disorders, amnesia, aggression, autism, vertigo, dementia, circadian rhythm disorders and gastric motility disorders.

The invention also provides a method of treating a condition which requires modulation of a dopamine receptor, which comprises administering to a mammal in need thereof an effective amount of a compound of formula (I) as hereinbefore described or a pharmaceutically acceptable salt or solvate thereof.

The invention also provides a method of treating schizophrenia, mood disorders, anxiety disorders, substance-related disorders, sleep disorders, eating disorders, autistic disorder, attenton-deficit/hyperactivity disorder, disruptive behaviour disorder, tic disorders, personality disorders, cognition impairment in other diseases, sexual dysfunction, Parkinson's disease, dyskinetic disorders, depression, bipolar disorder, cognitive impairment, obesity, emesis, movement disorders, obsessive-compulsive disorders, amnesia, aggression, vertigo, dementia and circadian rhythm disorders which comprises administering to a mammal in need thereof an effective amount of a compound of formula (I) as hereinbefore described or a pharmaceutically acceptable salt or solvate thereof.

The invention also provides a method of treating psychotic disorders, schizophrenia, Parkinson's disease, substance abuse, dyskinetic disorders, depression, bipolar disorder, anxiety, cognitive impairment, eating disorders, obesity, sexual dysfunction, sleep disorders, emesis, movement disorders, obsessive-compulsive disorders, amnesia, aggression, autism, vertigo, dementia, circadian rhythm disorders and gastric motility disorders which comprises administering to a mammal in need thereof an effective amount of a compound of formula (I) as hereinbefore described or a pharmaceutically acceptable salt or solvate thereof.

A preferred use for dopamine antagonists according to the present invention is in the treatment of psychotic disorders, schizophrenia, Parkinson's disease, substance abuse, dyskinetic disorders, depression, bipolar disorder, anxiety and cognitive impairment.

“Treatment” includes prophylaxis, where this is appropriate for the relevant condition(s).

It will be appreciated by those skilled in the art that the compounds according to the invention may advantageously be used in conjunction with one or more other therapeutic agents, for instance 5HT₃ antagonists, serotonin agonists, NK-1 antagonists, selective serotonin reuptake inhibitors (SSRI), noradrenaline re-uptake inhibitors (SNRI), non-selective reuptake inhibitors of one or more of serotonin, noradrenaline and norepinephrine, CRF-1 antagonists, tricyclic antidepressants, dopaminergic antidepressants, H₃ antagonists, 5HT_{1A} antagonists, 5HT_{1B} antagonists, 5HT_{1D} antagonists, 5HT₄ partial agonists, D₁ agonists, M₁ agonists and/or anticonvulsant agents. The compounds according to the invention may also advantageously be used in conjunction with cyclooxygenase-2 (COX-2) inhibitors.

It will be appreciated that the compounds of the combination or composition may be administered simultaneously (either in the same or different pharmaceutical formulations), separately or sequentially.

Suitable 5HT₃ antagonists which may be used in combination of the compounds of the inventions include for example ondansetron, granisetron and metoclopramide.

Suitable serotonin agonists which may be used in combination with the compounds of the invention include sumatriptan, rauwolscine, yohimbine and metoclopramide.

Suitable SSRIs which may be used in combination with the compounds of the invention include fluoxetine, citalopram, femoxetine, fluvoxamine, paroxetine, indalpine, sertraline and zimeldine.

Suitable SNRIs which may be used in combination with the compounds of the invention include venlafaxine and reboxetine.

Suitable tricyclic antidepressants which may be used in combination with a compound of the invention include imipramine, amitriptiline, chlomipramine and nortriptiline.

Suitable dopaminergic antidepressants which may be used in combination with a compound of the invention include bupropion and amineptine.

Suitable anticonvulsant agents which may be used in combination of the compounds of the inventions include for example divalproex, carbamazepine and diazepam.

Suitable COX-2 inhibitors include rofecoxib (available under the tradename VIOXX®, from Merck, U.S. Pat. No. 5,474,995); celecoxib (available under the tradename CELEBREX®, from Pfizer, U.S. Pat. No. 5,466,823); valdecoxib (available under the tradename BEXTRA®, from Pfizer, U.S. Pat. No. 6,633,272); etoricoxib (available under the tradename ARCOXIA®, from Merck, U.S. Pat. No. 5,861,419); lumiracoxib (available under the tradename PREXIGE®, from Novartis); paracoxib (U.S. Pat. No. 5,932,598); COX-189 from Novartis; BMS347070 from Bristol Myers Squibb; tiracoxib (JTE522) from Japan Tobacco; ABT963 from Abbott; CS502 from Sankyo; 2-(4-ethoxyphenyl)-3-(3-methanesulfonylphenyl)-pyrazolo[1,5-b]pyridazine (GlaxoSmithKline) and 2-butoxy-4-[4-(methylsulfonyl)phenyl]-6-(trifluoromethyl)pyrimidine (GlaxoSmithKline).

The compounds of formula (I) and their pharmaceutically acceptable salts and solvates thereof are also suitable for combination with other typical and atypical antipsychotics to provide improved treatment of psychotic disorders. Particular advantages associated with the combinations, uses and methods of treatment of compounds of formula (I) and their pharmaceutically acceptable salts and solvates thereof include equivalent or improved efficacy at doses of administration which are lower than those commonly used for the individual components. Improved treatments of positive symptoms and/or negative symptoms and/or cognitive symptoms of the psychotic disorder may also be observed. The combinations, uses and methods of treatment of the invention may also provide advantages in treatment of patients who fail to respond adequately or who are resistant to treatment with certain antipsychotic agents.

The combination therapies of the invention are preferably administered adjunctively. By adjunctive administration is meant the coterminous or overlapping administration of each of the components in the form of separate pharmaceutical compositions or devices. This regime of therapeutic administration of two or more therapeutic agents is referred to generally by those skilled in the art and herein as adjunctive therapeutic administration; it is also known as add-on therapeutic administration. Any and all treatment regimes in which a patient receives separate but coterminous or overlapping therapeutic administration of the compounds of formula (I) or a pharmaceutically acceptable salt or solvate thereof and at least one antipsychotic agent are within the scope of the current invention. In one embodiment of adjunctive therapeutic administration as described herein, a patient is typically stabilised on a therapeutic administration of one or more of the of the components for a period of time and then receives administration of another component. The compounds of formula (I) or a pharmaceutically acceptable salt or solvate thereof may be administered as adjunctive therapeutic treatment to patients who are receiving administration of at least one antipsychotic agent, but the scope of the invention also includes the adjunctive therapeutic administration of at least one antipsychotic agent to patients who are receiving administration of compounds of formula (I) or a pharmaceutically acceptable salt or solvate thereof.

The combination therapies of the invention may also be administered simultaneously. By simultaneous administration is meant a treatment regime wherein the individual components are administered together, either in the form of a single pharmaceutical composition or device comprising or containing both components, or as separate compositions or devices, each comprising one of the components, administered simultaneously. Such combinations of the separate individual components for simultaneous combination may be provided in the form of a kit-of-parts.

In a further aspect therefore, the invention provides a method of treatment of a psychotic disorder by adjunctive therapeutic administration of compounds of formula (I) or a pharmaceutically acceptable salt or solvate thereof to a patient receiving therapeutic administration of at least one antipsychotic agent. In a further aspect, the invention provides the use of compounds of formula (I) or a pharmaceutically acceptable salt or solvate thereof in the manufacture of a medicament for adjunctive therapeutic administration for the treatment of a psychotic disorder in a patient receiving therapeutic administration of at least one antipsychotic agent. The invention further provides compounds of formula (I) or a pharmaceutically acceptable salt or solvate thereof for use for adjunctive therapeutic administration for the treatment of a psychotic disorder in a patient receiving therapeutic administration of at least one antipsychotic agent.

In a further aspect, the invention provides a method of treatment of a psychotic disorder by adjunctive therapeutic administration of at least one antipsychotic agent to a patient receiving therapeutic administration of compounds of formula (I) or a pharmaceutically acceptable salt or solvate thereof. In a further aspect, the inventifon provides the use of at least one antipsychotic agent in the manufacture of a medicament for adjunctive therapeutic administration for the treatment of a psychotic disorder in a patient receiving therapeutic administration of compounds of formula (I) or a pharmaceutically acceptable salt or solvate thereof. The invention further provides at least one antipsychotic agent for adjunctive therapeutic administration for the treatment of a psychotic disorder in a patient receiving therapeutic administration of compounds of formula (I) or a pharmaceutically acceptable salt or solvate thereof.

In a further aspect, the invention provides a method of treatment of a psychotic disorder by simultaneous therapeutic administration of compounds of formula (I) or a pharmaceutically acceptable salt or solvate thereof in combination with at least one antipsychotic agent. The invention further provides the use of a combination of compounds of formula (I) or a pharmaceutically acceptable salt or solvate thereof and at least one antipsychotic agent in the manufacture of a medicament for simultaneous therapeutic administration in the treatment of a psychotic disorder. The invention further provides the use of compounds of formula (I) or a pharmaceutically acceptable salt thereof in the manufacture of a medicament for simultaneous therapeutic administration with at least one antipsychotic agent in the treatment of a psychotic disorder. The invention further provides compounds of formula (I) or a pharmaceutically acceptable salt thereof for use for simultaneous therapeutic administration with at least one antipsychotic agent in the treatment of a psychotic disorder. The invention further provides the use of at least one antipsychotic agent in the manufacture of a medicament for simultaneous therapeutic administration with compounds of formula (I) or a pharmaceutically acceptable salt thereof in the treatment of a psychotic disorder.

In further aspects, the invention provides a method of treatment of a psychotic disorder by simultaneous therapeutic administration of a pharmaceutical composition comprising compounds of formula (I) or a pharmaceutically acceptable salt or solvate thereof and at least one mood stabilising or antimanic agent, a pharmaceutical composition comprising compounds of formula (I) or a pharmaceutically acceptable salt or solvate thereof and at least one mood stabilising or antimanic agent, the use of a pharmaceutical composition comprising compounds of formula (I) or a pharmaceutically acceptable salt or solvate thereof and at least one mood stabilising or antimanic agent in the manufacture of a medicament for the treatment of a psychotic disorder, and a pharmaceutical composition comprising compounds of formula (I) or a pharmaceutically acceptable salt or solvate thereof and at least one mood stabilising or antimanic agent for use in the treatment of a psychotic disorder.

In a further aspect, the invention provides a kit-of-parts for use in the treatment of a psychotic disorder comprising a first dosage form comprising compounds of formula (I) or a pharmaceutically acceptable salt or solvate thereof and one or more further dosage forms each comprising a antipsychotic agent for simultaneous therapeutic administration.

Within the context of the present invention, the term psychotic disorder includes those disorders mentioned above, such as schizophrenia, mood disorders, anxiety disorders. substance-related disorders, sleep disorders, eating disorders, autistic disorder, attention-deficit/hyperactivity disorder, disruptive behaviour disorder, tic disorders, personality disorders, cognition impairment in other diseases, sexual dysfunction, dyskinetic disorders, depression, bipolar disorder, cognitive impairment and obsessive-compulsive disorders and all the various forms of the disorders as mentioned herein. which are contemplated as part of the present invention.

Examples of antipsychotic drugs that are useful in the present invention include, but are not limited to: butyrophenones, such as haloperidol, pimozide, and droperidol; phenothiazines, such as chlorpromazine, thioridazine, mesoridazine, trifluoperazine, perphenazine, fluphenazine, thiflupromazine, prochlorperazine, and acetophenazine; thioxanthenes, such as thiothixene and chlorprothixene; thienobenzodiazepines; dibenzodiazepines; benzisoxazoles; dibenzothiazepines; imidazolidinones; benzisothiazolyl-piperazines; triazine such as lamotrigine; dibenzoxazepines, such as loxapine; dihydroindolones, such as molindone; aripiprazole; and derivatives thereof that have antipsychotic activity.

Examples of tradenames and suppliers of selected antipsychotic drugs that are suitable for use in the present invention are as follows: clozapine (available under the tradename CLOZARIL®, from Mylan, Zenith Goldline, UDL, Novartis); olanzapine (available under the tradename ZYPREXA®, from Lilly; ziprasidone (available under the tradename GEODON®, from Pfizer); risperidone (available under the tradename RISPERDAL®, from Janssen); quetiapine fumarate (available under the tradename SEROQUEL®, from AstraZeneca); sertindole (available under the tradename SERLECT®); amisulpride (available under the tradename SOLION®, from Sanofi-Synthelabo); haloperidol (available under the tradename HALDOL®, from Ortho-McNeil); haloperidol decanoate (available under the tradename HALDOL decanoate®); haloperidol lactate (available under the tradenames HALDOL® and INTENSOL®) chlorpromazine (available under the tradename THORAZINE®, from SmithKline Beecham (GSK); fluphenazine (available under the tradename PROLIXIN®, from Apothecon, Copley, Schering, Teva, and American Pharmaceutical Partners, Pasadena); fluphenazine decanoate (available under the tradename PROLIXIN decanoate®); fluphenazine enanthate (available under the tradename PROLIXIN®); fluphenazine hydrochloride (available under the tradename PROLIXIN®); thiothixene (available under the tradename NAVANE®;, from Pfizer); thiothixene hydrochloride (available under the tradename NAVANE®); trifluoperazine (10-[3-(4-methyl-1-piperazinyl)propyl]-2-(trifluoromethyl)phenothiazine dihydrochloride, available under the tradename STELAZINE®, from SmithKlien Beckman; perphenazine (available under the tradename TRILAFON®; from Schering); perphenazine and amitriptyline hydrochloride (available under the tradename ETRAFON TRILAFON®); thioridazine (available under the tradename MELLARIL®; from Novartis, Roxane, HiTech, Teva, and Alpharma); molindone (available under the tradename MOBAN®, from Endo); molindone hydrochloride (available under the tradename MOBAN®); loxapine (available under the tradename LOXITANE®; from Watson); loxapine hydrochloride (available under the tradename LOXITANE®); and loxapine succinate (available under the tradename LOXITANE®). Furthermore, benperidol (Glianimon®), perazine (Taxilan®) or melperone (Eunerpan®)) may be used.

Other suitable antipsychotic drugs include promazine (available under the tradename SPARINE®), triflurpromazine (available under the tradename VESPRIN®), chlorprothixene (available under the tradename TARACTAN®), droperidol (available under the tradename INAPSINE®), acetophenazine (available under the tradename TINDAL®;), prochlorperazine (available under the tradename COMPAZINE®), methotrimeprazine (available under the tradename NOZINAN®), pipotiazine (available under the tradename PIPOTRIL®), iloperidone, pimozide and flupenthixol.

In one further aspect of the invention, suitable antipsychotic agents include olanzapine, risperidone, quetiapine, aripiprazole, haloperidol, clozapine, ziprasidone and osanetant.

For use in medicine, the compounds of the present invention are usually administered as a standard pharmaceutical composition. The present invention therefore provides in a further aspect a pharmaceutical composition comprising a compound of formula (I) as hereinbefore described or a pharmaceutically (i.e. physiologically) acceptable salt thereof and a pharmaceutically (i.e. physiologically) acceptable carrier. The pharmaceutical composition can be for use in the treatment of any of the conditions described herein.

The compounds of formula (I) may be administered by any convenient method, for example by oral, parenteral (e.g. intravenous), buccal, sublingual, nasal, rectal or transdermal administration and the pharmaceutical compositions adapted accordingly.

The compounds of formula (I) as hereinbefore described and their pharmaceutically acceptable salts which are active when given orally can be formulated as liquids or solids, for example syrups, suspensions or emulsions, tablets, capsules and lozenges.

A liquid formulation will generally consist of a suspension or solution of the compound or pharmaceutically acceptable salt in a suitable liquid carrier(s) for example an aqueous solvent such as water, ethanol or glycerine, or a non-aqueous solvent, such as polyethylene glycol or an oil. The formulation may also contain a suspending agent, preservative, flavouring or colouring agent.

A composition in the form of a tablet can be prepared using any suitable pharmaceutical carrier(s) routinely used for preparing solid formulations. Examples of such carriers include magnesium stearate, starch, lactose, sucrose and cellulose.

A composition in the form of a capsule can be prepared using routine encapsulation procedures. For example, pellets containing the active ingredient can be prepared using standard carriers and then filled into a hard gelatin capsule; alternatively, a dispersion or suspension can be prepared using any suitable pharmaceutical carrier(s), for example aqueous gums, celluloses, silicates or oils and the dispersion or suspension then filled into a soft gelatin capsule.

Typical parenteral compositions consist of a solution or suspension of the compound or pharmaceutically acceptable salt in a sterile aqueous carrier or parenterally acceptable oil, for example polyethylene glycol, polyvinyl pyrrolidone, lecithin, arachis oil or sesame oil. Alternatively, the solution can be lyophilised and then reconstituted with a suitable solvent just prior to administration.

Compositions for nasal administration may conveniently be formulated as aerosols, drops, gels and powders. Aerosol formulations typically comprise a solution or fine suspension of the active substance in a pharmaceutically acceptable aqueous or non-aqueous solvent and are usually presented in single or multidose quantities in sterile form in a sealed container, which can take the form of a cartridge or refill for use with an atomising device. Alternatively the sealed container may be a unitary dispensing device such as a single dose nasal inhaler or an aerosol dispenser fitted with a metering valve which is intended for disposal once the contents of the container have been exhausted. Where the dosage form comprises an aerosol dispenser, it will contain a propellant which can be a compressed gas such as compressed air or an organic propellant such as a fluorochlorohydrocarbon. The aerosol dosage forms can also take the form of a pump-atomiser.

Compositions suitable for buccal or sublingual administration include tablets, lozenges and pastilles, wherein the active ingredient is formulated with a carrier such as sugar and acacia, tragacanth, or gelatin and glycerin.

Compositions for rectal administration are conveniently in the form of suppositories containing a conventional suppository base such as cocoa butter.

Compositions suitable for transdermal administration include ointments, gels and patches. Preferably the composition is in unit dose form such as a tablet, capsule or ampoule.

Each dosage unit for oral administration contains preferably from 1 to 250 mg (and for parenteral administration contains preferably from 0.1 to 25 mg) of a compound of the formula (I) or a pharmaceutically acceptable salt thereof calculated as the free base.

The pharmaceutically acceptable compounds of the invention will normally be administered in a daily dosage regimen (for an adult patient) of, for example, an oral dose of between 1 mg and 500 mg, preferably between 10 mg and 400 mg, e.g. between 10 and 250 mg or an intravenous, subcutaneous, or intramuscular dose of between 0.1 mg and 100 mg, preferably between 0.1 mg and 50 mg, e.g. between 1 and 25 mg of the compound of the formula (I) or a pharmaceutically acceptable salt thereof calculated as the free base, the compound being administered 1 to 4 times per day. Suitably the compounds will be administered for a period of continuous therapy, for example for a week or more.

No toxicological effects are indicated or expected when a compound of the invention is administered in the above mentioned dosage range.

Biological Test Methods

Binding Experiments on Cloned Doramine (e.g. D2 and D3) Receptors

The ability of the compounds to bind selectively to human D2/D3 dopamine receptors can be demonstrated by measuring their binding to cloned receptors. The inhibition constants (Kᵢ) of test compounds for displacement of [¹²⁵I]-lodosulpride binding to human D2/D3 receptors expressed in CHO cells were determined as follows. The cell lines were shown to be free from bacterial, fungal and mycoplasmal contaminants, and stocks of each were stored frozen in liquid nitrogen. Cultures were grown as monolayers or in suspension in standard cell culture media. Cells were recovered by scraping (from monolayers) or by centrifugation (from suspension cultures), and were washed two or three times by suspension in phosphate buffered saline followed by collection by centrifugation. Cell pellets were stored frozen at −80° C. Crude cell membranes were prepared by homogenisation followed by high-speed centrifugation, and characterisation of cloned receptors achieved by radioligand binding.

Preparation of CHO cell membranes: Cell pellets were gently thawed at room temperature, and resuspended in about 20 volumes of ice-cold Extraction buffer; 5 mM EDTA, 50 mM Trizma pre-set crystals (pH7.4@37° C.), 1 mM MgCl₂, 5 mM KCl and 120 mM NaCl. The suspension was homogenised using an Ultra-Turrax at full speed for 15 seconds. The homogenate was centrifuged at 18,000 r.p.m for 15 min at 4° C. in a Sorvall RC5C centrifuge. Supernatant was discarded, and homogenate re-suspended in extraction buffer then centrifugation was repeated. The final pellet was resuspended in 50 mM Trizma pre-set crystals (pH 7.4@37° C.) and stored in 1 ml aliquot tubes at −80° C. (D2=3.0E+08 cells, D3=7.0E+07 cells and D4=1.0E+08 cells). The protein content was determined using a BCA protocol and bovine serum albumin as a standard (Smith, P. K., et al., Measurement of protein using bicinchoninic acid. Anal. Biochem. 150, 76-85 (1985)).

Binding experiments: Crude D2/D3 cell membranes were incubated with 0.03 nM [¹²⁵I]-lodosulpride (˜2000 Ci/mmol; Amersham, U. K., and the test compound in a buffer containing 50 mM Trizma pre-set crystals (pH 7.4@37° C.), 120 mM NaCl, 5 mM KCl, 2 mM CaCl₂, 1 mM MgCl₂, 0.3% (w/v) bovine serum albumin. The total volume is 0.2 ml and incubated in a water bath at 37° C. for 40 minutes. Following incubation, samples were filtered onto GF/B Unifilters using a Canberra Packard Filtermate, and washed four times with ice-cold 50 mM Trizma pre-set crystals (pH 7.4@37° C.). The radioactivity on the filters was measured using a Canberra Packard Topcount Scintillation counter. Non-specific binding was defined with 10 μM SKF-102161 (YM-09151). For competition curves, 10 serial log concentrations of competing cold drug were used (Dilution range: 10 μM-10 μpM). Competition curves were analysed using Inflexion, an iterative curve fitting programme in Excel. Results were expressed as pKᵢ values where
pKᵢ=−log10[Ki].

The exemplified compounds have pKᵢ values within the range of 6.3-8.9 at the dopamine D₃ receptor.

The exemplified compounds have pKᵢ values within the range of 5.6-8.5 at the dopamine D₂ receptor.

Binding Experiments on Cloned 5-HT₆ Receptors

Compounds can be tested following the procedures outlined in WO 98/27081.

The exemplified compounds have pKᵢ values within the range of 7.2-10.0 at the serotonin 5-HT₆ receptor.

Binding Experiments on Cloned 5-HT_{2A} and 5-HT_{2C} Receptors

Compounds can be tested following the procedures outlined in WO 94/04533.

The exemplified compounds have pK₁ values within the range of 7.0-9.9 at the serotonin 5-HT_{2C} receptor and 7.5-9.9 at the serotonin 5-HT_{2A} receptor.

The invention is further illustrated by the following non-limiting examples:

Description 1

### 7-Methoxy-3-methyl-2,3,4,5-tetrahydro-1H-3-benzazepine (D1)

A mixture of 7-methoxy-2,3,4,5-tetrahydro-1H-3-benzazepine hydrochloride (see EP 285287) (25 g, 125 mmol) and 37% formalin (25 mL)in dichloroethane (250 mL) was treated with sodium triacetoxyborohydride (30 g, 250 mmol) keeping the internal temperature below 20° C. After stirring for 2 h, water was added and the pH adjusted to 10 using 50% sodium hydroxide solution. The organic layer was separated, dried over sodium sulfate and evaporated to dryness to afford the product D1 (23 g).

Description 2

### 8-Methoxy-3-methyl-2,3,4,5-tetrahydro-1H-3-benzazepine-7-sulfonyl fluoride (D2)

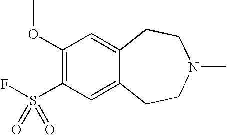

### a) 7-Methoxy-3-methyl-2,3,4,5-tetrahydro-1H-3-benzazepine (D2a)

A mixture of 7-methozy-2,3,4,5-tetrahydro-1H-3-benzazepine hydrochloride (see EP 285287) (25 g, 125 mmol) and 37% formalin (25 mL)in dichloroethane (250 mL) was treated with sodium triacetoxyborohydride (30 g, 250 mmol) keeping the internal temperature below 20° C. After stirring for 2 h, water was added and the pH adjusted to 10 using 50% sodium hydroxide solution. The organic layer was separated, dried over sodium sulfate and evaporated to dryness to afford the product D2a (23 g).

### b) 8-Methoxy-3-methyl-2,3,4,5-tetrahydro-1H-3-benzazepine-7-sulfonic acid (D2b)

The product from part a) (23 g) was dissolved in trifluoroacetic acid (125 mL), and then stirred in an ice bath while chlorosulfonic acid (16.5 mL, 250 mmol) was added dropwise. The solution was stirred for 30 min, then evaporated to dryness to afford the title sulfonic acid D2b which was used directly in the next step.

### c) 8-Methoxy-3-methyl-2,3,4,5-tetrahydro-1H-3-benzazepine-7-sulfonyl chloride (D2c)

The sulfonic acid from part b) was dissolved in thionyl chloride (75 mL) and the solution refluxed for 30 min. After cooling, the solution was evaporated to dryness to afford the title sulfonyl chloride D2c which was used directly in the next step.

### d) 8-Methoxy-3-methyl-2,3,4,5-tetrahydro-1H-3-benzazepine-7-sulfonyl fluoride (D2)

The sulfonyl chloride from part c) was dissolved in acetonitrile (500 mL) and potassium fluoride (37 g, 625 mmol) and 18-crown-6 (1 crystal) added. The mixture was stirred for 18 h, then quenched with cold aqueous sodium bicarbonate solution until the pH equalled 8. The mixture was extracted twice with ethyl acetate, washed with bicarbonate solution then brine, dried and evaporated to afford the sulfonyl fluoride D1 (25 g).

Description 3

### 7-[6-(tert-Butyl-dimethylsilyloxymethyl)pyridine-3-sulfonyl]-8-methoxy-3-methyl-2,3,4,5-tetrahydro-1H-3-benzazepine (D3)

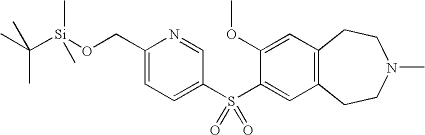

To a solution of 5-Bromo-2-(tert-butyl-dimethylsilyloxymethyl)pyridine (4.61 g, 15.3 mmol, 3.0 eq) in anhydrous THF (40 ml) under argon at −78° C. was added dropwise n-butyllithium (6.1 ml 2.5M in THF, 15.3 mmol, 3.0 eq). When addition was complete, stirring was continued for 10 mins at −78° C. then a solution of D2 (1.39 g, 5.1 mmol, 1.0 eq) in anhydrous THF (15 ml) was added dropwise over 10 mins. When addition was complete the red/purple solution was allowed to warm to −30° C. and stirred for a further 2 h before quenching at this temperature with acetic acid (2 ml) followed by water (20 ml). The mixture was allowed to warm to room temperature and partitioned between saturated NaHCO₃ solution (300 ml) and ethyl acetate (200 ml). The aqueous phase was extracted with more ethyl acetate (200 ml) and the combined organic phase washed with water (300 ml) and brine (300 ml) and dried over anhydrous MgSO₄. Concentration in vacuo gave an oil. Purification by flash column chromatography over silica, eluting with 0-5% MeOH in CH₂Cl₂ afforded the title compound D3 as a pale yellow solid (1.50 g, 62%). MH⁺ 477. ¹H NMR δ (CDCl₃) 0.04 (6H, s), 0.87 (9H, s), 2.27 (3H, s), 2.47 (4H, m), 2.86 (4H, m), 3.69 (3H, s), 4.79 (2H, s), 6.56 (1H, s) 7.56 (1H, d), 7.76 (1H, s), 8.15 (1H, dd), 8.95 (1H, d).

Description 4

### [5-(8-Methoxy-3-methyl-2,3,4,5-tetrahydro-1H-3-benzazepine-7-sulfonyl)pyridin-2-yl]methanol (D4)

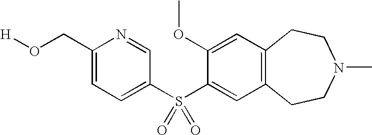

A solution of the silyl ether D3 (1.36 g, 2.85 mmol, 1.0 eq) in THF (10 ml) and 5M HCl (30 ml) was stirred at room temperature for 1 h then evaporated to dryness in vacuo. The residue was purified on an SCX cartridge, first eluting the impurities/excess acid with methanol and then the product eluted with 1M NH₃ in MeOH. The methanolic ammonia solution was evaporated to dryness, affording the title compound D4 as a pale yellow solid (1.02 g, 100%). MH⁺ 363. ¹H NMR δ (CDCl₃) 2.37 (3H, s), 2.56 (4H, m), 2.94 (4H, m), 3.78 (3H, s) 4.84 (2H, s), 6.66 (1H, s), 7.40 (1H, d), 7.84 (1H, s), 8.21 (1H, dd), 9.01 (1H, d).

### EXAMPLE 1

### 7-[(5-{[(4-Fluorophenyl)oxy]methyl}-2-thienyl)sulfonyl]-3-methyl-8-(methyloxy)-2,3,4,5-tetrahydro-1H-3-benzazepine (E1)

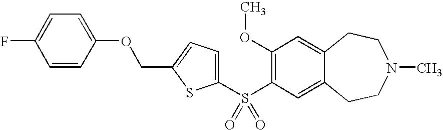

### a) 7-[(5-bromo-2-thienyl)sulfonyl]-3-methyl-8-(methyloxy)-2,3,4,5-tetrahydro-1H-3-benzazepine (E1a)

A solution of 3-methyl-7-(methyloxy)-2,3,4,5-tetrahydro-1H-3-benzazepine D1 (400 mg, 2.0 mmol) in trifluoroacetic acid (4 ml) and trifluoromethanesulfonic acid (0.4 ml) was treated with 5-bromo-2-thiophenesulfonyl chloride (785 mg, 3.0 mmol) and indium (III) chloride (40 mg, 0.2 mmol). The mixture was stirred at 80° C. for 30 minutes, then at room temperature for 10 hours. It was neutralised with 2N sodium hydroxide and extracted with dichloromethane (2×20 ml). The combined organics were concentrated in vacuo and the residue was applied to a pre-wetted SCX cartridge which was eluted with methanol followed by ammonia in methanol. The ammonia/methanol fraction was concentrated in vacuo and the residue was purified by silica chromatography eluting with 5% methanol in dichloromethane to give the desired product E1a (200 mg, 24%). MH⁺ 416/418. ¹H NMR δ (CDCl₃) 2.40 (s, 3H), 2.45-2.65 (m, 4H), 2.85-3.00 (m, 4H), 3.90 (s, 3H), 6.70 (s, 1H), 7.00 (d, 1H), 7.50 (d, 1H), 7.75 (s, 1H).

### b) 5-{[3-methyl-8-(methyloxy)-2,3,4,5-tetrahydro-1H-3-benzazepin-7-yl]sulfonyl}-2-thiophenecarbaldehyde (E1b)

A solution of E1a (200 mg, 0.48 mmol) in dry tetrahydrofuran (2 ml) was stirred under argon and cooled to −78°. t-Butyllithium (0.63 ml, 1.5M in , 0.95 mmol) was added dropwise. After stirring for 2 minutes, N,N-dimethylformamide (0.2 ml) was added and the mixture was stirred at −78° for 1 hour. Water was added and the mixture was extracted with dichloromethane. The organics were concentrated in vacuo and purified by silica chromatography eluting with 3 then 4% methanol in dichloromethane. This gave the desired product E1b (90 mg, 51%). MH⁺ 366. ¹H NMR δ (CDCl₃) 2.37 (s, 3H), 2.50-2.65 (m, 4H), 2.80-3.00 (m, 4H) 3.90 (s, 3H) 6.70 (s, 1H), 7.67 (d, 1H), 7.75-7.80 (m, 2H), 9.95 (s, 1H).

### c) (5-{[3-methyl-8-(methyloxy)-2,3,4,5-tetrahydro-1H-3-benzazepin-7-yl]sulfonyl}-2-thienyl)methanol (E1c)

A solution of E1b (90 mg, 0.25 mmol) in methanol (2 ml) was treated with sodium borohydride (11.4 mg, 0.30 mmol) and stirred for 1 hour at room temperature. The mixture was then treated with water (10 ml) and dichloromethane (10 ml) and the phases were separated. The organics were concentrated in vacuo and the residue was purified by silica chromatography eluting with 5 then 10% methanol in dichloromethane. This gave the desired product E1c (80 mg, 99%). MH⁺ 368. ¹H NMR δ (CDCl₃) 2.35 (s, 3H), 2.50-2.65 (m, 4H) 2.85-3.00 (m, 4H), 3.90 (s, 3H), 4.85 (s, 2H), 6.70 (s, 1H), 6.95 (d, 1H), 1H).

### 7-[(5-{[(4-fluorophenyl)oxy]methyl)-2-thienyl)sulfonyl]-3-methyl-8-(methyloxy)-2,3,4,5-tetrahydro-1H-3-benzazespinee (E1)

A solution of E2c (80 mg, 0.22 mmol), triphenylphosphine (115 mg, 0.44 mmol) and 4-fluorophenol (49 mg, 0.44 mmol) in tetrahydrofuran (2 ml) was stirred under argon and treated with diisopropyl azodicarboxylate (0.08 ml, 0.44 mmol) dropwise. The reaction mixture was stirred for 12 hours and was then evaporated to dryness. The residue was applied in methanol to a pre-wetted SCX cartridge and eluted with methanol followed by ammonia in methanol. The ammonia/methanol fraction was concentrated in vacuo and purified by silica chromatography eluting with 4% methanol in dichloromethane to give the desired product E1(5 mg, 5%). MH⁺ 462. ¹H NMR δ (CDCl₃) 2.37 (s, 3H), 2.45-2.65 (m, 3H) 2.8-3.0 (m, 4H), 3.86 (s, 3H), 5.17 (s, 2H), 6.70 (s, 1H), 6.85-6.90 (m, 2H) 6.95-7.05 (m, 3H) 7.65 (d, 1H), 7.79 (s,1H).

### EXAMPLE 2

### 7-({5-[(4-Chlorophenyl)oxy]-2-thienyl}sulfonyl)-3-methyl-8-(methyloxy)-2,3,4,5-tetrahydro-1H-3-benzazepine (E2)

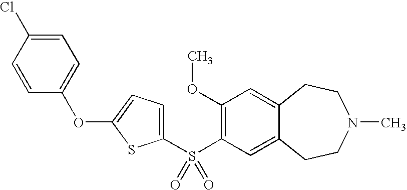

A solution of 7-[(5-bromo-2-thienyl)sulfonyl]-3-methyl-8-(methyloxy)-2,3,4,5-tetrahydro-1H-3benzazepine (E1a, 150 mg, 0.36 mmol) and 4-chlorophenol (70 mg, 0.54 mmol) in N,N-dimethylformamide (3 ml) was treated with potassium carbonate (100 mg, 0.72 mmol) and the mixture was heated in a sealed tube in a microwave reactor at 150° C. for 15 minutes. The mixture was diluted with dichloromethane and washed with saturated sodium bicarbonate solution. The organics were separated and concentrated in vacuo to give a residue which was purified by silica chromatography eluting with a gradient of 0 to 10% methanol in dichloromethane. The product had to be further purified using Mass-Directed Auto Prep and SCX to give the title compound E2 as a white solid (20 mg, 12%). MH⁺ 464/466. ¹H NMR δ (CDCl₃) 2.20 (s, 3H0, 2.50-2.65 (m, 4H), 2.90-3.00 (m, 4H), 3.90 (s, 3H), 6.40 (d, 1H), 6.70 (s, 1H), 7.05-7.10 (m, 2H), 7.30-7.35 (m, 2H), 7.53 (d, 1H), 7.75 (s, 1H).

### EXAMPLE 3

### 7-({5-[(4-Fluorophenyl)methyl]-2-thienyl}sulfonyl)-3-methyl-8-(methyloxy)-2,3,4,5-tetrahydro-1H-3-benzazepine (E3)

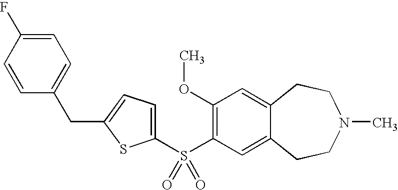

### a) (5-Bromo-2-thienyl)(4-fluorophenyl)methanol (E3a)

A stirred solution of 5-bromo-2-thiophenecarboxaldehyde (1.0 g, 5.2 mmol) in dry tetrahydrofuran (20 ml) under argon was cooled to 0° C. and 4-fluorophenylmagnesium bromide solution (6.3 ml, 1M in tetrahydrofuran, 6.3 mmol) was added. The mixture was allowed to warm to room temperature and was stirred for 4 hours. It was then poured into a solution of Rochelles salt and stirred for 30 minutes. The mixture was extracted with ethyl acetate and the combined organics were washed with saturated sodium bicarbonate solution and dried. Evaporation in vacuo gave E3a as a brown oil (1.76 g) which was taken directly to the next step without further purification.

### b) 2-Bromo-5-[(4-fluorophenyl)methyl]thiophene (E3b)

A solution of E3a (1.76 g crude) and triethyl silane (4.9 ml, 30.7 mmol) in chloroform was stirred at 0° C. under argon and trifluoromethanesulfonic acid (1.1 ml, 12.4 mmol) was added dropwise. The mixture was stirred at room temperature for 1 hour, then it was diluted with dichloromethane (50 ml) and washed with saturated sodium bicarbonate solution. The organic phase was dried and concentrated in vacuo to give a gum which was purified by silica chromatography eluting with 40/60 petroleum ether followed by hexane. Evaporation of the appropriate fractions gave the desired product E3b (596 mg, 42%).

### 7-({5-[(4-Fluorophenyl)methyl]-2-thienyl}sulfonyl-3-methyl-8-(methyloxy)-2,3,4,5-tetrahydro-1H-3-benzazepine (E3)

A solution of E3b (596 mg, 2.2 mmol) in dry tetrahydrofuran (10 ml) was stirred under argon and cooled to −78° C. n-Butyllithium (1.0 ml, 2.5M in hexanes, 2.5 mmol) was added dropwise and the mixture was stirred at −78° C. for 15 minutes. A solution of 3-methyl-8-(methyloxy)-2,3,4,5-tetrahydro-1H-3benzazepine-7-sulfonyl fluoride D2 (200 mg, 0.73 mmol) in dry tetrahydrofuran (5 ml) was then added dropwise, and the reaction was stirred at −78° C. for 1 hour, before being quenched by the addition of acetic acid (5 ml). The mixture was allowed to warm to room temperature and then treated with saturated sodium bicarbonate solution. It was extracted with ethyl acetate and the organics were concentrated in vacuo to give a residue which was purified by silica chromatography eluting with dichloromethane. Product required further purification so it was submitted to Mass-Directed Auto Prep to give the desired product E3 as an off-white solid (40 mg). MH⁺ 446. ¹H NMR δ (CD₃OD) 2.70 (s, 3H), 2.95-3.18 (m, 8H), 3.85 (s, 3H), 4.20 (s, 2H), 6.88 (d, 1H), 6.95-7.08 (m, 3H), 7.25 (dd, 2H), 7.58 (d, 1H), 7.77 (s, 1H).

### EXAMPLE 4

### 7-[6-(4-Chlorophenoxymethyl)pyridine-3-sulfonyl]-8-methoxy-3-methyl-2,3,4,5-tetrahydro-1H-3-benzazepine (E4)

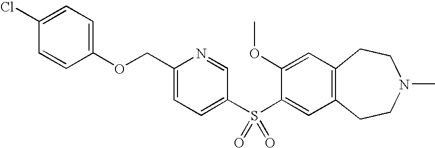

To a stirred solution of alcohol D4 (0.100 g, 0.276 mmol, 1.0 eq), 4-chlorophenol (0.039 g, 0.303 mmol, 1.1 eq) and triphenylphosphine (0.079 g, 0.303 mmol, 1.1 eq) in anhydrous THF (2 ml) at 0° C. was added dropwise dilsopropyl azodicarboxylate (60 μl, 0.061 g, 0.303 mmol, 1.1 eq). The resultant solution was stirred at room temperature for 16 h then water (15 ml) was added and the mixture extracted with ethyl acetate (2×10 ml). The combined organic phase was washed with water (20 ml), brine (20 ml), dried over anhydrous MgSO₄ and concentrated to dryness If vacuo. The residual oil was purified first on an SCX cartridge, eluting the impurities/excess reagents with methanol and then the product eluted with 1M NH₃ in MeOH and evaporated. A second purification by flash column chromatography over silica, eluting with 0-10% MeOH in CH₂Cl₂ afforded the title compound E4 as a pale yellow solid (0.094 g, 72%). MH⁺ 473/475. ¹H NMR δ (CDCl₃) 2.37 (3H, s), 2.56 (4H, m), 2.94 (4H, m), 3.78 (3H, s), 5.28 (2H, s), 6.66 (1H, s), 6.89 (2H, d), 7.25 (2H, d), 7.65 (1H, d) 7.85 (1H, s), 8.28 (1H, dd), 9.12 (1H, d).

### EXAMPLE 5

### 7-[6-(3,4-Difluorophenoxymethyl)pyridine-3-sulfonyl]-8-methoxy-3-methyl-2,3,4,5-tetrahydro-1H-3-benzazepine (E5)

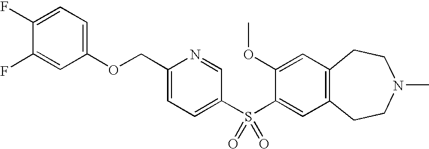

To a stirred solution of alcohol D4 (0.106 g, 0.292 mmol, 1.0 eq), 3,4-difluorophenol (0.042 g, 0.322 mmol, 1.1 eq) and triphenylphosphine (0.084 g, 0.322 mmol, 1.1 eq) in anhydrous THF (2 ml) at 0° C. was added dropwise diisopropyl azodicarboxylate (63 μl, 0.065 g, 0.322 mmol, 1.1 eq). The resultant solution was stirred at room temperature for 16 h then water (15 ml) was added and the mixture extracted with ethyl acetate (2×10 ml). The combined organic phase was washed with water (20 ml), brine (20 ml), dried over anhydrous MgSO₄ and concentrated to dryness in vacuo. The residual oil was purified first on an SCX cartridge, eluting the impurities/excess reagents with methanol and then the product eluted with 1M NH₃ in MeOH and evaporated. A second purification by flash column chromatography over silica, eluting with 0-10% MeOH in CH₂Cl₂ afforded the title compound E5 as a pale yellow solid (0.060 g, 43%), MH⁺ 475. ¹H NMR δ (CDCl₃) 2.37 (3H, s), 2.56 (4H, m), 2.95 (4H, m), 3.78 (3H, s), 5.19 (2H, s), 6.65 (2H, m), 6.79 (1H, m), 7.07 (1H, q), 7.62 (2H, d), 7.85 (1H, s) 8.29 (1H, dd), 9.12 (1H, d).

All ¹H NMR are consistent with the structures shown.

All of the compounds listed below in Table 1 relate to compounds of formula (IF):

TABLE 1

| | | | | | | (IF) |
|---|---|---|---|---|---|---|
| 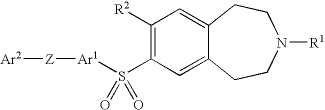 | | | | | | |
| Example | R¹ | R² | Ar¹ | Ar² | Z | MH⁺ |
| 1 | Me | OMe | thien-2-yl | 4-F-phenyl | —O—CH₂— | 462 |
| 2 | Me | OMe | thien-2-yl | 4-Cl-phenyl | —O— | 465 |
| 3 | Me | OMe | thien-2-yl | 4-F-phenyl | —CH₂— | 446 |
| 4 | Me | OMe | pyrid-3-yl | 4-Cl-phenyl | —O—CH₂— | 473/ |
| | | | | | | 475 |
| 5 | Me | OMe | pyrid-3-yl | 3,4-diF- | —O—CH₂— | 475 |
| | | | | phenyl | | |

All publications, including but not limited to patents and patent applications, cited in this specification are herein incorporated by reference as if each individual pub location were specifically and individually indicated to be incorporated by reference herein as though fully set forth.

What is claimed is:

1. A compound of formula (I): 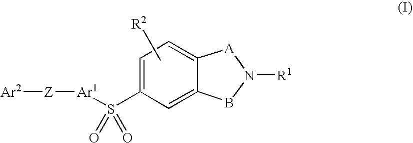
wherein
A and B represent the groups —(CH₂)ₘ— and —(CH₂)ₙ— respectively;
R¹ represents hydrogen or C₁₋₆alkyl;
R² represents hydrogen, halogen, hydroxy, cyano, nitro, hydroxyC₁₋₆alkyl, trifluoromethyl, trifluoromethoxy, C₁₋₆alkyl, C₁₋₆alkoxy, C₁₋₆fluoroalkoxy, —(CH₂)ₚC₃₋₆cycloalkyl, —(CH₂)ₚOC₃₋₆cycloalkyl, —COC₁₋₆alkyl, —SO₂C₁₋₆alkyl, —SOC₁₋₆alkyl, —S— C₁₋₆alkyl, —CO₂C₁₋₆alkyl, —CO₂NR³R⁴, —SO₂NR³R⁴, —(CH₂)ₚNR³R⁴, —(CH₂)ₚNR³COR⁴, optionally substituted aryl ring, optionally substituted heteroaryl ring, a fused bicyclic heteroaromatic ring system or optionally substituted heterocyclyl ring;
Ar¹ represents optionally substituted heteroaryl ring;
Ar² represents optionally substituted aryl ring or optionally substituted heteroaryl ring;
Z represents —(CH₂)_{q}X— wherein the —(CH₂)_{q}— group is attached to Ar², or —X(CH₂)_{q}— wherein X is attached to Ar², and wherein any of the —CH₂— groups may be optionally substituted by one or more C₁₋₆alkyl groups;
X represents oxygen, —CH(OR⁵)—, —NR⁵— or —CH₂— wherein the —CH₂— group may be optionally substituted by one or more C₁₋₆alkyl groups;
R³ and R⁴ each independently represent hydrogen, C₁₋₆alkyl or, together with the nitrogen or other atoms to which they are attached, form an azacycloalkyl ring or an oxo-substituted azacycloalkyl ring;
R⁵ represents hydrogen or C₁₋₆alkyl;
m and n independently represent an integer selected from 1 and 2;
p independently represents an integer selected from 0, 1, 2 and 3;
q independently represents an integer selected from 0, 1, 2 and 3;
or a pharmaceutically acceptable salt or solvate thereof,
with the proviso that when Ar¹ is a pyridyl group, and Z is —CH₂X— where X is attached to the Ar¹ group, X is selected from —CH(OR⁵)—, —NR⁵— and —CH₂— wherein the —CH₂— group may be optionally substituted by one or more C₁₋₆alkyl groups.

2. A compound of formula (I) according to claim 1 which is
7-[(5-{[(4-Fluorophenyl)oxy]methyl}-2-thienyl)sulfonyl]-3-methyl-8-(methyloxy)-2,3,4,5-tetrahydro-1H-3-benzazepine;
7-({5-[(4-Chlorophenyl)oxy]-2-thienyl}sulfonyl)-3-methyl-8-(methyloxy)-2,3,4,5-tetrahydro-1H-3-benzazepine;
7-({5-[(4-Fluorophenyl)methyl]-2-thienyl } sulfonyl)-3-methyl-8-(methyloxy)-2,3,4,5-tetrahydro-1H-3-benzazepine;
7-[6-(4-Chlorophenoxymethyl)pyridine-3-sulfonyl]-8-methoxy-3-methyl-2,3,4,5-tetrahydro-1H-3-benzazepine;
7-[6-(3,4-Difluorophenoxymethyl)pyridine-3-sulfonyl]-8-methoxy-3-methyl-2,3,4,5-tetrahydro-1H-3-benzazepine,
and pharmaceutically acceptable salts and solvates thereof.

3. A pharmaceutical composition comprising a compound of formula (I) as claimed in claim 1 or a pharmaceutically acceptable salt thereof and a pharmaceutically acceptable carrier therefor.

4. (canceled)

5. (canceled)

6. (canceled)

7. A method of treating a condition which requires modulation of a dopamine receptor, which comprises administering to a mammal in need thereof an effective amount of a compound of formula (I) or a pharmaceutically acceptable salt or solvate thereof as claimed in claim 1.

8. A method of treating psychotic disorders, schizophrenia, Parkinson's disease, substance abuse, dyskinetic disorders, depression, bipolar disorder, anxiety and cognitive impairment, which comprises administering to a mammal in need thereof an effective amount of a compound of formula (I) or a pharmaceutically acceptable salt or solvate thereof as claimed in claim 1.
